(19) United States
(12) Patent Application Publication
(10) Pub. No.: US 2010/0292086 A1
(43) Pub. Date: Nov. 18, 2010

(54) METHOD OF SCREENING FOR METABOLITE TRANSPORT MODIFIERS

(75) Inventor: Alan Kleinfeld, La Jolla, CA (US)

Correspondence Address: KNOBBE MARTENS OLSON & BEAR LLP, 2040 MAIN STREET, FOURTEENTH FLOOR, IRVINE, CA, 92614, US

(73) Assignee: Membrane Sciences, LLC, San Diego, CA (US)

(21) Appl. No.: 12/863,765

(22) PCT Filed: Jan. 27, 2009
(86) PCT No.: PCT/US09/32166

§ 371 Date: Jul. 20, 2010

Related U.S. Application Data
(60) Provisional application No. 61/024,362, filed on Jan. 29, 2008.

(51) Int. Cl. C40B 30/00; G01N 33/566

(52) U.S. Cl. 506/7; 435/7.21

(57) ABSTRACT

Methods for discovering compounds that modulate metabolite transport across cell membranes are disclosed. Fluorescently labeled protein probes which have an affinity for an unbound metabolite are trapped in cells. The influx of the unbound metabolite into the cell causes a change in the fluorescence of the protein probe. The ability of test compounds to modulate the fluorescent signal is used to screen for compounds which affect transport of the unbound metabolite. In an example, the unbound metabolite is an unbound free fatty acid and the probe is a fatty acid binding protein.

### RELATED APPLICATIONS

This application claims priority to U.S. Provisional Application No. 61/024,362, filed Jan. 29, 2008, which is incorporated herein by reference.

### STATEMENT REGARDING FEDERALLY SPONSORED R&D

This work was supported in part by Grant No. R01DK058762 from the National Institute of Health. Consequently, the U.S. government may have certain rights to this invention.

### BACKGROUND OF THE INVENTION

### Field of the Invention

The field of the invention relates to a high throughput method for discovering compounds that modulate the transport of metabolites across cell membranes.

For purposes of the present disclosure, fatty acids are non esterified carboxylated alkyl chains of 1-30 carbons atoms which may exist as neutral (e.g. protonated, sodium or potassium salt) or ionic species, depending upon the pH and conditions of the aqueous media. Free fatty acids (FFA) are equivalent to fatty acids and both terms refer to the totality of FFA including those in aqueous solution as monomers plus those that are not in solution (for example bound to other macromolecules (proteins, membranes), cells or part of an aggregate of FFA (micelles, soaps and other more complex aggregates). FFA present as monomers in aqueous solution (either charged or neutral) are referred to as unbound free fatty acids (FFAu).

For purposes of the present disclosure, intracellular lipid binding proteins (iLBP) are a family of low-molecular weight single chain polypeptides. There are four recognized subfamilies. Subfamily I contains proteins specific for vitamin A derivatives such as retinoic acid and retinol. Subfamily II contains proteins with specificities for bile acids, eiconsanoids, and heme. Subfamily III contains intestinal type fatty acid binding proteins (FABPs) and Subfamily IV contains all other types of fatty acid binding protein (Haunerland, et al. (2004) Progress in Lipid Research vol. 43: 328-349). The entire family is characterized by a common 3-dimensional fold. Ligand binding properties of the different subfamilies overlap considerably. The wild type proteins of subfamilies I (Richieri et al (2000) Biochemistry 39:7197-7204) and II both bind fatty acids and those of subfamily II bind fatty acids as well as their native ligands. Moreover, single amino acid substitutions are able to interconvert the ligand binding properties of proteins of subfamilies I and II (Jakoby et al (1993) Biochemistry 32:872-878).

For the purposes of the present disclosure FFAu probes are iLBPs that are fluorescently labeled and that undergo a change in the ratio of a fluorescence index measured at 2 different wavelengths upon binding a FFA. Such probes may be used to determine the aqueous concentration of FFAu which is otherwise difficult because of their poor solubility properties in aqueous solutions. A change in the ratio of the fluorescence response is essential for the accurate determination of the intracellular as well as extracellular concentrations of unbound analytes.

FFA, particularly the long chain FFA, serve a variety of essential functions; they provide a major portion of physiologic energy needs (Neely, J. R. et al (1974) Ann. Rev. Physio. 36: 413-459), are important constituents in the synthesis of complex lipids and play critical roles in cell signaling (Distel, R. J. et al (1992) J Biol. Chem. 267:5937-5941). Most FFA are obtained from circulating plasma FFA, the levels of which are regulated by the rate at which FFA are generated, predominantly by adipose tissue (Nielsen, S. et al (2003) J. Clin. Invest 111:981-988), and the rate at which they are metabolized, predominantly by liver, muscle and adipose tissue. Regulation of circulating FFA levels is critical because FFA play essential roles in homeostasis; deviations from normal levels reflect pathology and may adversely affect health (Apple, F. S. et al (2004) Clinical Proteomics 1:41-44; Boden, G. (2002) Curr. Opin. Clin Nutr. Metab Care 5:545-549; Jouven, X. et al (2001) Circulation 104:756-761; Kleinfeld, A. M. et al (1996) Amer. J. Cardiol. 78:1350-1354; Kleinfeld, A. M. et al (1997) Biochemistry 36:5702-5711; Kleinfeld, A. M. et al (2002) Am Coll Cardiol 39:312A; Kurien, V. A. et al (1966) The Lancet July 16:122-127; Makiguchi, M. et al (1991) Cardiovasc Drugs and Ther 5:753-761; Oliver, M. F. (2002) Am J Med 112:305-311; Opie, L. H. (1975) Amer. J. Cardiol. 36:938-953; Soloff, L. A. (1970) American Heart Journal 80:671-674).

Although most FFA in circulation are bound to albumin (Richieri, G. V. et al (1993) Biochemistry 32:7574-7580; Spector, A. A. (1975) J. Lipid Res. 16:165-179), the small fraction of unbound FFA (FFAu) in the aqueous phase mediates physiologic activity and is most sensitive to changes in health and disease (Apple, F. S. et al (2004) Clinical Proteomics 1:41-44; Richieri, G. V. et al (1995) J. Lipid Res. 36:229-240; Yuvienco, J. M. et al (2005) Am. J. Perinatol 22:429-436). Moreover, the FFAu, not FFA-albumin complexes, are transported across membranes.

In the course of their storage, production and consumption, FFA must cross the plasma and mitochondrial membranes of many different cells. Because these membranes might be involved in the regulation of FFA trafficking, the mechanism by which FFA are transported across membranes has been an area of considerable interest. Although FFA transport across membranes has been the subject of many studies there remains considerable disagreement about the mechanism by which FFA are transported across membranes. Among other issues this controversy stems from a lack of agreement about the need for membrane proteins in cellular transport and the function of the several membrane proteins that have been reported to play a role in the cellular uptake of FFA.

Several suggested mechanisms of FFA membrane transport have emerged from these studies. These include the proposal that transport of long chain FFA does not involve a membrane transport protein but rather occurs by rapid and passive transport through the lipid phase of a membrane (Kamp, F. et al (1995) Biochemistry 34:11928-11937; Thomas, R. M. et al (2002) Biochemistry 41:1591-1601; Zhang, F. et al (1996) Biochemistry 35:16055-16060). On the other hand are reports that have identified four different proteins that in some way facilitate FFA transport in mammalian cells: FABPpm (Schwieterman, W. et al (1988) Proc. Natl. Acad. Sci USA 85:359-363), CD36/FAT (Abumrad, N. A. et al (1993) J. Biol. Chem. 268:17665-17668), FATP (Schaffer, J. E. et al (1994) Cell 79:427-436) and caveolin (Trigatti, B. L. et al (1999) Biochem Biophys. Res Commun. 255; 34-39).

Compounds that modulate transport are important for identifying transport proteins and are potentially important as therapeutic agents. Several such compounds have been reported to inhibit transport in studies that have characterized putative transport proteins including phloretin, DIDS, SSO and others (Abumrad, N. A. et al (1981) J. Biol. Chem. 256:9183-9191; Abumrad, N. A. et al (1983) Federation Proceedings 42:1975; Harmon, C. M. et al (1991) J. Membrane Biol. 121:261-268). However, other studies have not confirmed the activity of these compounds (Faergeman, N. J. et al (2001) J Biol. Chem. 276:37051-37059; Kampf, J. P. et al (2004) J. Biol. Chem. 279:35775-35780). More recently it has been shown that the methods used previously to study FFA transport, and that were used to identify transport proteins and inhibitors, were in fact not sensitive to the transport of FFA across membranes (Kampf, J. P. et al (2004) J. Biol. Chem. 279:35775-35780; Kampf, J. P. et al (2007) Physiology 22:7-29; Kleinfeld, A. M. et al (2004) J. Am. Soc. Mass Spectrom. 15:1572-1580). As a consequence it is likely that neither the FFA transport proteins nor the inhibitors were correctly identified (Kampf, J. P. et al (2007) Am. J. Physiol Endocrinol. Metab).

The methods used in the studies that identified specific proteins as FFA transporters or identified inhibitors involved FFA uptake. FFA uptake involves the determination of the amount of radioactive or fluorescent fatty acid associated with or that accumulates in the whole cell. For example this is the method described in Black et al (U.S. Pat. No. 7,070,944) for identifying modulators of FFA uptake. The problem with measuring uptake is that in such measurements much of the FFA transported into the cells is removed in the wash step that is used before measuring the amount of cell associated FFA (Kampf, J. P. et al (2004) J. Biol. Chem. 279:35775-35780; Kleinfeld, A. M. et al (2004) J. Am. Soc. Mass Spectrom. 15:1572-1580). Thus what is detected in such measurements is the radioactive or fluorescent metabolic products of the added FFA rather than the FFA itself. Therefore, modulators discovered by such methods do not affect the transport but rather affect downstream processes such as esterification or metabolism. As such, compounds found by monitoring uptake are not modulators of the membrane transport step in the transport of FFA across cell membranes. The method of discovering compounds that affect the membrane transport step is the field of this invention.

To overcome the limitations imposed by uptake measurements, methods to investigate FFA transport across plasma membranes directly have been developed (Kampf, J. P. et al (2004) J. Biol. Chem. 279:35775-35780; Kleinfeld, A. M. et al (2004) J. Am. Soc. Mass Spectrom. 15:1572-1580). These methods allow FFA influx to be monitored without having to remove extracellular FFA by washing, as is required in the uptake measurements. This is important because most of the intracellular FFA that is transported into cells, and retains the chemical identity of a FFA, can be rapidly extracted by removing extracellular FFA (Kampf, J. P. et al (2004) J. Biol. Chem. 279:35775-35780; Kleinfeld, A. M. et al (2004) J. Am. Soc. Mass Spectrom. 15:1572-1580). Methods have been described to monitor the changes in the cytosolic unbound FFA concentration in living cells in response to changes in the extracellular unbound FFA concentration using adipocytes or preadipocytes microinjected with ADIFAB, acrylodan labeled rat intestinal fatty acid binding protein (Kampf, J. P. et al (2004) J. Biol. Chem. 279:35775-35780; Kampf, J. P. et al (2007) Am. J. Physiol Endocrinol. Metab)).

Because the method of microinjection of individual cells is not suitable for high throughput screening for compounds that might modulate FFA transport, a method is needed in which intracellular FFAu levels can be monitored accurately in large numbers of cells. Such a method would allow intracellular FFAu levels to be monitored in the presence and absence of a compound to be tested as a potential modulator (a test compound) under conditions in which the extracellular FFAu levels can be changed by well defined magnitudes. Furthermore, it should be possible to configure this method so that the cells, test compounds and FFAu can be formatted into arrays so that large numbers of tests can be carried out efficiently. The subject invention satisfies these requirements by using novel methods.

For the purposes of the present disclosure “probes” are proteins that are fluorescently labeled and that undergo a change in fluorescence upon binding a metabolite. In preferred embodiments the probe undergoes a change in the ratio of a fluorescence index measured at different 2 wavelengths upon binding a metabolite. Such probes may be used to determine the aqueous concentration of specific unbound analytes including FFA, metabolites and other lipophilic hormones, and drugs, which is otherwise difficult because of their poor solubility properties in aqueous solutions. A change in the ratio of the fluorescence response is essential for the accurate determination of the intracellular as well as extracellular concentrations of unbound analytes.

### SUMMARY OF THE INVENTION

Embodiments of the invention are directed to methods for discovering compounds that modulate metabolite transport across cell membranes which include one or more of the following steps:
(a) trapping a probe in cells;
(b) contacting the cells in (a) with a test compound or a mixture of test compounds;
(c) measuring the fluorescence response of the trapped probe in the absence of added unbound metabolite;
(d) adding a defined level of unbound metabolite to the cells plus test compound or mixtures of test compounds of step (b);
(e) measuring the fluorescence response of the trapped probe in the presence of the test compounds and unbound metabolite;
(f) adding a macromolecule that provides a sink for metabolite and thereby reduces unbound metabolite to levels indistinguishable from those in (c);
(g) measuring the fluorescence response of the trapped probe in the presence of the test compounds and absence of unbound metabolite; and
(h) determining a change in influx and/or efflux an/or intracellular level or magnitude of the transported metabolite by comparing the fluorescence response measured in steps (c), (e), and (g), thereby identifying a potential modulator from the test compounds or mixture of test compounds.

Preferably, the probe is trapped in the cells by electroporation, use of lipid or peptide transfection reagents, or mechanical membrane disruption such as scrape, scratch, bead, or syringe loading.

Preferably, the cells are dispersed in multi-well plates and the fluorescence measurements are performed using a high throughput plate scanning fluorometer or microscope.

Preferably, the potential modulator discovered using a mixture of test compounds is then re-measured using the individual test compounds in the mixture.

In some preferred embodiments, the potential modulator is tested to determine if it affects the metabolite influx rate constants by measuring an influx time course of the metabolite.

In some preferred embodiments, the potential modulator is tested to determine if it affects the metabolite efflux rate constants by measuring an efflux time course of the metabolite.

In some preferred embodiments, the potential modulator is tested to determine if it affects the intracellular level or magnitude of metabolite transport by measuring the ratio of the intracellular to extracellular unbound metabolite concentrations.

Preferably, the potential modulator is tested to determine if it is fluorescent and whether its fluorescence interferes with the probe fluorescence.

Embodiments of the invention are directed to methods for discovering compounds that inhibit metabolite transport across cell membranes which include one or more of the following steps:
(a) adding cells, test compounds, mixtures of test compounds and combinations thereof to the wells of a multi-well plate;
(b) adding probe to the wells of (a);
(c) measuring the fluorescence response of each probe in each well in the absence of added unbound metabolite, wherein the fluorescence responses are measured under one or more of the following conditions:
   (i) the fluorescence response of the probe;
   (ii) the fluorescence response of the probe+cell,
   (iii) the fluorescence response of the probe+test compound or mixture of test compounds,
   (iv) the fluorescence response of the probe+cell+test compound or mixture of test compounds;
(d) adding a composition comprising a weakly buffered complex of the unbound metabolite and a carrier macromolecule to each well so that the level of unbound metabolite decreases in wells with cells that do not contain test compounds, (c)(ii), as compared to wells without cells, (c)(i), (c)(iii);
(e) measuring the fluorescence response of each probe in each well in the presence of added unbound metabolite from step (d), wherein the fluorescence responses are measured under one or more of the following conditions:
   (i) the fluorescence response of the probe+unbound metabolite;
   (ii) the fluorescence response of the probe+cell+unbound metabolite,
   (iii) the fluorescence response of the probe+test compound or mixture of test compounds+unbound metabolite,
   (iv) the fluorescence response of the probe+cell+test compound or mixture of test compounds+unbound metabolite;
(f) comparing the fluorescence responses of step (c) to the fluorescence response of step (e), thereby identifying potential modulators.

In some preferred embodiments of the methods described above, the potential modulator is tested to determine if the test compound interacts with the probe and either blocks the unbound metabolite's interaction with the probe or induces a probe response or changes the probe response to the unbound metabolite.

In some preferred embodiments of the methods described above, the potential modulator is tested to determine whether the test compound interacts with the carrier macromolecule so as to alter the unbound metabolite-carrier interaction and/or alter the interaction of the test compound with the unbound metabolite transporter.

In some preferred embodiments of the methods described above, the potential modulator is tested to determine whether the test compound affects cellular lipolysis and/or unbound metabolite metabolism in such a way as to have the appearance of affecting unbound metabolite transport.

In some preferred embodiments of the methods described above, the probe is ADIFAB and the unbound metabolite is FFAu.

Further aspects, features and advantages of this invention will become apparent from the detailed description of the preferred embodiments which follow.

### DETAILED DESCRIPTION OF THE PREFERRED EMBODIMENT

While the described embodiment represents the preferred embodiment of the present invention, it is to be understood that modifications will occur to those skilled in the art without departing from the spirit of the invention. The scope of the invention is therefore to be determined solely by the appended claims.

Embodiments of the invention are directed to methods of discovering compounds that modulate the influx and/or efflux of metabolites through cell membranes. Such modulators are expected to have important applications for treatment of a range of diseases. For purposes of the present disclosure, metabolites are physiologically important molecules whose molecular weight is approximately 2000 Da or less. These include molecules that occur naturally in the course of human or animal physiology or pathophysiology, and drug molecules and their metabolic products and nutrient molecules and their metabolic products. A fraction of each metabolite is present as monomers in aqueous solution (either charged or neutral). We refer to this fraction as the unbound metabolite. Many metabolites are hydrophobic molecules with low aqueous solubility and unbound concentrations that are much lower than their “total” concentration, where the bulk of the “total” may be bound to proteins or cells. In preferred embodiments, the unbound metabolite is an unbound free fatty acid (FFAu).

Besides free fatty acids, possible metabolites include but are not limited to molecules such as drugs, drug metabolites, hormones, prostaglandins, leukotrienes, sphingosine, sphingolipids, phospholipids, glycolipids, cholesterol and cholesterol derivatives and other steroids, lipid-soluble vitamins, bile salts, enzyme cofactors, retinoids such as retinoic acid and retinal, heme and heme metabolites, amino acids, peptides, carbohydrates and multivalent ions.

In principle virtually any type of cell can be used. Preferably, the cells are mammalian cells. Some preferred cell types include adipocytes, cardiac myocytes, hepatocytes, muscle cells, erythrocytes, enterocytes, endothelial cells and neuronal cells. Cell culture techniques for culture and manipulation of a wide range of cell types are well known.

In a most preferred embodiment, a fibroblast cell line, the 3T3F442A pre-adipocytes, is used. These cells are grown in Dulbecco's modified Eagles' medium (Sigma-Aldrich, St. Louis, Mo.) with 10% calf serum, 1 mM sodium pyruvate and 3.7 mg/ml sodium bicarbonate, pH 7.4 (CS Media). Cells are split up to four times after seeding. Adherent preadipocytes are harvested using trypsin, washed with phosphate-buffered saline (PBS) and collected by centrifugation (250×g for 5 min) prior to syringe loading.

Preferably, when the cells are removed from the culture flasks they are washed carefully to remove trypsin because trypsin will cleave an acrylodan containing peptide from the probe and result in an increase in the fluorescence ratio (R) in the absence of unbound metabolite. In a preferred embodiment, cells are also washed with BSA to remove endogenous unbound metabolite and then washed twice more with C-HEPES (20 mM HEPES, 140 mM NaCl, 5 mM KCl, 1 mM MgSO₄, 1 mM CaCl₂, 1 mM Na₂HPO₄ and 5.5 mM glucose at pH 7.4) to remove all traces of BSA.

The washed cells are loaded with a probe. For the purposes of the present disclosure, probes are fluorescently labeled proteins that reveal a measurable change in fluorescence upon binding to unbound metabolite. The described method is applicable to any unbound metabolite which can bind to a probe and produce a fluorescence change. In preferred embodiments, these probes are intracellular lipid binding proteins (iLBP) or a mutein of an iLBP. More preferably the probes are fatty acid binding proteins (FABP) or FABP muteins. The muteins are iLBPs or FABPs in which one or more native amino acid residues have been replaced with a non-native amino acid residue, thereby affecting the ability of the iLBP or FABP to bind an unbound metabolite. In preferred embodiments, mutation involves one or more amino acid substitutions in the binding cavity or the helical cap of a FABP. Such probes are described in U.S. application Ser. No. 11/085,792, Kleinfeld (U.S. Pat. No. 5,470,714) and Huber et al (Huber, H. A. et al (2006) Biochemistry 45:14263-14274) which are incorporated herein by reference. In a preferred embodiment, the probe is ADIFAB which is a rat intestinal FABP labeled with acrylodan, and the unbound metabolite is FFAu. In the preferred embodiment the probe ADIFAB is trapped inside the cells and used to detect the change in intracellular FFAu levels.

The probes have the characteristic that their fluorescence changes in a measurable way when they bind metabolites. The ability of each such probe to respond to a particular metabolite can then be assessed by measuring the change in fluorescence upon addition of defined concentrations of the unbound metabolite.

Any method of trapping probes inside the cells may be used including but not limited to: electroporation, use of lipid or peptide transfection reagents, or mechanical membrane disruption as in scrape, scratch, bead, or syringe loading. In a preferred embodiment, cells are syringe-loaded preadipocytes with ADIFAB. Syringe-loaded preadipocytes are prepared essentially by the method described by Clarke and McNiel (Clarke, M. S. et al (1992) J. Cell Sci. 102 (Pt 3):533-541). A confluent, 75 cm² flask of 3T3F442A preadipocytes (approximately 12 million cells total) are trypsinized, collected by centrifugation (250×g for 5 minutes) and resuspended in 150 μl of 400 μM ADIFAB in Buffer-0 (20 mM HEPES, 140 mM NaCl, 5 mM KCl, 1 mM Na₂HPO₄, and 5.5 mM glucose at pH 7.4). While on ice or at 4° C., the cell suspension is passed through a 30-gauge needle approximately 20 times and the total volume of the suspension is approximately 250 ml which consists of 150 μl ADIFAB plus about 100 μl of cell pellet. The cells are then re-sealed by adding about 1 mM CaCl₂ and about 1 mM MgSO₄ and are then allowed to incubate on ice for 10 minutes before centrifugation at 11,000×g for about 5 min at 4° C. Dead cells are removed by: resuspending the cell pellet in calf serum (CS) media, adding the cell suspension to a cell culture flask containing CS media, incubating at 37° C. for 1-2 hours, aspirating the media containing the dead cells in suspension and then collecting the viable, adherent, cells.

The measurement of transport of the metabolite in the loaded cells may be performed using a fluorescence spectrophotometer, a fluorescence flow cytometer, a quantitative fluorescence microscope or any instrument capable of measuring the fluorescence response of the trapped probes such as ADIFAB. In a preferred embodiment a fluorescence plate reader, a fluorescence spectrophotometer equipped with a device capable of measuring the fluorescence in each well of a multi-well plate is used to measure the fluorescence change upon binding of an unbound metabolite by the probe. The plates may be composed of any material suitable for cell fluorescence measurements and may be of any number of wells including but not limited to 96, 384 and 1536.

In a preferred embodiment, the first step for monitoring the effect of test compounds on transport of metabolites is the addition of aliquots of the cell suspension containing unbound metabolite-probe to the wells of a multi-well plate. Some wells in the plate may contain probe only and some wells may contain cell suspension without trapped probe. In a next step one or more (mixture) of test compounds is added while mixing to wells containing the cell suspension, wells containing the probe only and wells containing buffer only. Different compounds and/or different mixtures of compounds are added to wells containing the cell suspension, wells containing the probe only and wells containing buffer only. In some embodiments, the test compounds may be added to the probes and/or trapped probes before addition of the unbound metabolites.

In a preferred embodiment the compounds to be tested for their ability to modulate transport are chosen from libraries of chemical compounds. Chemical libraries may range in size from 1 to several million compounds. The compounds may be drugs, natural products, chemicals synthesized by combinatorial chemistry methods or compounds synthesized to optimize a lead compound discovered by the screening methods of the inventive method. Compounds may be small (about 500 kDa or less) molecules, peptides of various molecular weights or antibodies. Libraries of compounds may be synthesized by well known methods of combinatorial chemistry or antibody production or may be obtained from commercial sources such as, for example, ASINEX, ChemBridge Corporation or Santa Cruz Biotechnology.

In a preferred second step, the probe's fluorescence indices in each well of the plate are measured. The indices may be fluorescence intensities, polarization or lifetimes. In a preferred embodiment the fluorescence intensities at two different fluorescence emission wavelengths are measured. For example, if the metabolite is FFA, the ratio of a FFAu-probe intensities at the two emission wavelengths is determined. These intensities may be corrected for the intensities of the cells without FFAu probe if necessary. These measurements allow the determination of the Ro value by the following formula:

Ro=F_{λ1}/F_{λ2}
wherein F_{λ1} is a measured fluorescence intensity (intensity of the cells with trapped probe minus intensity of the cells without trapped probe) at a first emission wavelength, F_{λ2} is a measured fluorescence intensity (intensity of the cells with trapped probe minus intensity of the cells without trapped probe) at a second emission wavelength.

This Ro is the R value in the absence of added FFA but in the presence of a test compound or mixtures thereof. In addition, from the appropriate wells, the fluorescence intensities of the test compounds, or mixtures, alone are determined and, also in appropriate wells, the effect of the test compounds on the FFAu probe alone is determined. These measurements allow the determination of the level of fluorescence from the test compound or mixtures only and allow the determination of the effect of the test compound or mixtures on the FFAu probe only.

In the third step of the preferred embodiment, unbound metabolite such as FFA is added to the wells of the plate while mixing. The unbound metabolite, such as FFA, is added as a complex with a carrier macromolecule that clamps the unbound metabolite level in each well at a well defined value. The carrier molecule can be any macromolecule that binds with the unbound metabolite non-covalently and includes but is not limited to a FFA binding protein such as albumin or FABP or a polymer such as cyclodextrin or a lipid vesicle. Clamping of the unbound metabolite level is determined by measuring with extracellular probe, in the wells containing the metabolite-carrier complex, the unbound metabolite levels in the absence and presence of added cells. Clamping is assured when the unbound metabolite level in presence of added cells is a predetermined level such as within 80%, preferably within 90%, more preferably within 95% or greater of the level in the absence of added cells. If the unbound metabolite level in the presence of added cells is less than the predetermined level in the absence of added cells, the concentration of macromolecule carrier is increased, maintaining the same ratio of metabolite to macromolecule carrier, until the unbound metabolite level in the presence of added cells is within the predetermined level.

In the fourth step the probe's fluorescence indices in each well of the plate are again measured. In the preferred embodiment the R value (ratio of the measured fluorescence indices) of the probe is determined and this is the R value of the probe trapped in the cells in the presence of unbound metabolite and test compound or mixtures. In addition the R value in the presence of unbound metabolite and test compound or mixtures is determined for the probe alone which allows the determination of the effect of the test compounds or mixtures on the ability of the probe to respond to unbound metabolite.

In the fifth step BSA (or other carrier macromolecule) without unbound metabolite (for example, FFA) is added to the wells of the plate while mixing and the probe's fluorescence indices in each well of the plate are again measured. In the preferred embodiment the R value of the probe is determined and this is the R value of the cells after unbound metabolite is removed while in the presence of the test compound or mixtures. In addition, the R value in the absence of unbound metabolite but in the presence of test compound or mixtures is determined for the probe alone. This allows the determination of the effect of the test compounds or mixtures on the ability of the probe to respond to the removal of unbound metabolite.

The results of the second to forth steps allow the determination of the effect of the test compound or mixtures on the ability of the cells to transport unbound metabolite such as FFA from outside to inside (influx). If the R value after unbound metabolite addition in the presence of test compound or mixtures is less than the R value in the absence of these compounds then these compounds may inhibit unbound metabolite influx. Alternatively, if the R value is larger in the presence as compared to the absence of these compounds then they may stimulate influx. Results from the second to fifth steps indicate whether the test compounds or mixtures inhibit efflux of unbound metabolite; an R value at step 5 that is larger than that at step 2 suggest inhibition.

If compounds are found to modulate influx or efflux and do not reveal interfering fluorescence and do not affect the probe response, then measurements are performed to determine whether an influx modulator increases or decreases influx rate constants and/or initial rates, alter steady state levels and thereby possibly affect the pumping of unbound metabolite into the cells. If a compound is found to affect efflux, then measurements are performed to determine whether an efflux modulator increases or decreases efflux rate constants and/or initial rates. Measurements are then performed to determine whether efflux rate constants are affected.

In an other embodiment of this invention compounds that inhibit metabolite transport (influx) into cells are screened by measuring the extracellular unbound metabolite levels using extracellular probes. In this embodiment metabolite-carrier complexes that are weakly buffered and therefore do not clamp the extracellular metabolite concentration are added to wells containing cells plus extracellular probe. In contrast to a strongly buffered metabolite-carrier complex, that clamps the unbound metabolite level so that it maintains a predetermined level in the absence and presence of cells, a weakly buffered complex allows the unbound metabolite level to decrease by 40%, preferably by 60%, more preferably by 80% or greater in the presence compared to the absence of added cells. If the unbound metabolite level in the presence of added cells decreases by less than the predetermined level in the absence of added cells, the concentration of macromolecule carrier is decreased, maintaining the same ratio of metabolite to macromolecule carrier, until the unbound metabolite level in the presence of added cells is reduced by the predetermined level. Under these conditions the unbound metabolite concentration decreases in the presence of transporting cells but is blocked or reduced in the presence of compound that inhibit influx.

In this embodiment test compounds can be screened for their ability to inhibit influx using intact cells with extracellular probe to monitor transport, without requiring the step of trapping probes within the cells.

In a preferred embodiment of this invention the methods may include one or more of the following steps:
(a) adding measuring buffer to all of the wells of a multi-well plate;
(b) adding cells to most of the wells of the multi-well plate;
(c) contacting most of the cells in (b) with different test compounds or mixtures of test compounds;
(d) adding probe to all the wells of (a);
(e) measuring the fluorescence response probe all wells in the absence of added unbound metabolite;
(f) adding unbound metabolite, using a weakly buffered complex of the metabolite and a carrier macromolecule so that the level of unbound metabolite decreases in wells with cells that do not contain test compounds, as compared to wells without cells;
(g) measuring the fluorescence response of the probe in all wells including those without cells, with cells but without test compound and cells contacted with test compounds or mixtures of test compounds and;
(h) determining whether the fluorescence changes from (e) to (g) are consistent with inhibition of influx by the test compounds.

### Applications

Compounds that block either influx or efflux of an unbound metabolite such as FFAu, could have significant therapeutic applications. For example, blocking FFAu efflux from adipocytes would reduce plasma FFA levels and might improve diabetes. In acute ischemia, reducing plasma FFA levels are expected to reduce death and recurrent myocardial infarctions. Reducing FFA efflux from human breast cancer tumors may improve the ability of cytotoxic T cells to clear the tumor. Compounds that block influx may help treat obesity.

### Example 1

### An Example of Screening from a Library of Compounds for their Ability to Modify FFA Influx and Efflux

### a) Preparation of Cells Containing ADIFAB.

3T3F442A preadipocytes were grown in Dulbecco's modified Eagles' medium with 10% calf serum (CS media) as described (Kampf, J. P. et al (2007) Am. J. Physiol Endocrinol. Metab). Approximately 10 million adherent preadipocytes were harvested using trypsin, washed with phosphate-buffered saline (PBS) and collected by centrifugation. The cells were re-suspended on ice in 150 μl of 400 μM ADIFAB in Buffer-O (20 mM HEPES, 140 mM NaCl, 5 mM KCl, 1 mM Na₂HPO₄, and 5.5 mM glucose at pH 7.4). While maintaining the cells at 4° C., the cell suspension was passed through a 30-gauge needle 18 times following which the cells were set on ice for 10 minutes. Resealing was initiated by adding 1 mM CaCl₂ plus 1 mM MgSO₄ and after another 10 minutes on ice, the syringe-loaded cells were pelleted by centrifugation (11,000×g for 5 min at 4° C.) to remove extracellular ADIFAB. These syringe loaded cells were then re-suspended in 1 ml of CS media, added to a 75 cm² cell culture flask containing 10 ml of CS media and incubated at 37° C. for approximately 2 hours. After this time most of the viable cells had adhered. The media containing the dead cells was removed from the flask and the viable cells were harvested using trypsin and centrifuged for 5 minutes at 250 g. Cells were washed in PBS, re-suspended in buffer 0 and placed on ice.

b) Dispensing Syringe Loaded Cells, Compounds to be Tested and Fatty Acids into Multi-Well Plates.

The cell suspension (at approximately 100,000 cells/ml) was warmed to approximately 22° C. and 200 μL of the cell suspension was added to the wells of a 96-well plate. Stock solutions of the compounds to be tested were 1 mM in DMSO and in this screen a mixture of two such compounds were tested in each well with the concentration for each compound set at 10 μM. Thus in each well DMSO was present at 2%. Oleic acid (OA) was added to wells as a complex with BSA so as to clamp the unbound OA concentration at a defined level. In this case the unbound OA concentration was approximately 100 nM and the BSA concentration was about 20 μM. For measuring efflux an additional 20 μM fatty acid free BSA was added to wells containing the OA-BSA complex.

### c) Fluorescence Measurements of the Multi-Well Plate.

Measurements of the fluorescence response of ADIFAB in the syringe loaded cells was carried out in 3 steps. First a 96-well plate loaded with cells and mixed with compounds to be tested was inserted into a MicroMax plate reader coupled to a SPEX Fluorolog 3 fluorometer from JY Horiba. This instrument was used to record the fluorescence intensities at 432 and 505 nm in each well. From these measurements the ratio of the 505 to 432 intensities were determined and these together with the individual intensities are shown in Table 1 as R(B) and I(B)505 and I(B)432 respectively. Second, the OA-BSA complex was added and mixed in each well and the 432 and 505 nm were again recorded (R(FA) and I(FA) in Table 1). Third, BSA without fatty acid was added and mixed in each well and the 432 and 505 nm were again recorded (R(BSA) and I(BSA) in Table 1).

### d) Interpretation of Results.

The results shown in Table 1 are the results of screening 50 pairs of test compounds (100 test compounds). The table also shows the results of averaging the R values and intensities over all the wells values. A second set of averages is also shown (−Fluor) and in these, contributions from test compounds that exhibit significant fluorescence are removed from the average. In this example one or both of 9 of the test pairs (18 compounds) of compounds exhibited significant fluorescence. The results show that after removal of these 9 cases, the average R value increases from an initial value of 0.293 to 0.737 upon adding OA and then returns to 0.296 upon adding BSA without OA. An examination of the 41 cases involving non-fluorescent compounds together with subsequent re-screening indicated that none of the 82 non-fluorescent compounds significantly affected either influx or efflux.

TABLE 1
Example of using 3T3F442A preadipocytes syringe loaded with ADIFAB to screen compounds for their ability to modify
FFA influx and efflux. Aliquots (200 μL) of the cells, suspended in a HEPES measuring buffer at pH 7.4, were
dispensed into wells of a 96-well plate (screening plate) and compounds from a library of compounds to be tested
were added to each well. The plate was scanned in a SPEX Fluorolog 3 fluorometer using a MicroMax plate reader
and the fluorescence intensities in each well at 432 and 505 nm were recorded, then again after adding oleic
acid as a complex with BSA and then again after addition of BSA without fatty acid. The individual intensities
and the ratios of 505 to 432 intensities, with cell blank and BSA fluorescence subtracted, are shown.

| | Screen | Screen | | | | | | | | | |
|---|---|---|---|---|---|---|---|---|---|---|---|
| ID | Plate | Well | R(B) | R(FA) | R(BSA) | I(B)505 | I(B)432 | I(FA)505 | I(FA)432 | I(BSA)505 | I(BSA)432 |
| 661 | 8 | F3 | 0.247 | 0.754 | 0.250 | 1092 | 4425 | 1836 | 2435 | 444 | 1775 |
| 662 | 8 | G3 | 0.247 | 0.831 | 0.239 | 1329 | 5382 | 1949 | 2345 | 450 | 1878 |
| 663 | 8 | H3 | 0.243 | 0.621 | 0.294 | 1206 | 4963 | 1753 | 2825 | 556 | 1887 |
| 664 | 8 | A4 | 0.253 | 0.735 | 0.279 | 1253 | 4962 | 2101 | 2857 | 340 | 1221 |
| 665 | 8 | B4 | 0.272 | 0.750 | 0.219 | 1549 | 5706 | 2366 | 3152 | 356 | 1627 |
| 666 | 8 | C4 | 0.255 | 0.690 | 0.229 | 1259 | 4942 | 2588 | 3752 | 369 | 1610 |
| 667 | 8 | D4 | 0.302 | 0.787 | 0.236 | 1197 | 3962 | 2116 | 2690 | 186 | 786 |
| 668 | 8 | E4 | 0.312 | 0.662 | 0.146 | 1342 | 4307 | 1577 | 2383 | 100 | 681 |
| 669 | 8 | F4 | 0.281 | 0.911 | 0.245 | 1561 | 5562 | 2136 | 2344 | 362 | 1475 |
| 670 | 8 | G4 | 0.261 | 0.564 | 0.428 | 1731 | 6629 | 2749 | 4878 | 1004 | 2347 |
| 671 | 8 | H4 | 0.255 | 0.656 | 0.266 | 1370 | 5375 | 2192 | 3340 | 326 | 1223 |
| 672 | 8 | A5 | 0.281 | 0.734 | 0.280 | 1269 | 4512 | 1982 | 2701 | 378 | 1350 |
| 673 | 8 | B5 | 0.671 | 0.676 | 0.618 | 54240 | 80833 | 43977 | 65047 | 12423 | 20096 |
| 674 | 8 | C5 | 0.254 | 0.617 | 0.591 | 1372 | 5405 | 2377 | 3854 | 420 | 709 |
| 675 | 8 | D5 | 0.378 | 0.754 | 0.389 | 1711 | 4525 | 2266 | 3005 | 384 | 986 |
| 676 | 8 | E5 | 0.293 | 0.819 | 0.313 | 1460 | 4983 | 2367 | 2889 | 329 | 1050 |
| 677 | 8 | F5 | 0.327 | 0.767 | 0.386 | 1586 | 4843 | 2619 | 3415 | 469 | 1215 |
| 678 | 8 | G5 | 0.128 | 0.140 | 0.099 | 1776 | 13842 | 2628 | 18791 | 929 | 9389 |
| 679 | 8 | H5 | 0.280 | 0.704 | 0.403 | 1510 | 5398 | 2346 | 3332 | 666 | 1652 |
| 680 | 8 | A6 | 0.259 | 0.286 | 0.205 | 7641 | 29469 | 11894 | 41558 | 5849 | 28485 |
| 681 | 8 | B6 | 0.548 | 0.871 | 0.718 | 3109 | 5672 | 3011 | 3457 | 1047 | 1458 |
| 682 | 8 | C6 | 0.267 | 0.446 | 0.231 | 2444 | 9141 | 3847 | 8634 | 558 | 2421 |
| 683 | 8 | D6 | 0.307 | 0.726 | 0.312 | 1442 | 4702 | 2404 | 3309 | 317 | 1016 |
| 684 | 8 | E6 | 0.353 | 0.839 | 0.312 | 1442 | 4084 | 2530 | 3016 | 331 | 1060 |
| 685 | 8 | F6 | 0.304 | 0.722 | 0.284 | 1567 | 5150 | 1774 | 2456 | 455 | 1605 |
| 686 | 8 | G6 | 0.506 | 1.154 | 0.320 | 1973 | 3902 | 2653 | 2299 | 374 | 1168 |
| 687 | 8 | H6 | 0.261 | 0.685 | 0.268 | 1497 | 5743 | 2094 | 3056 | 457 | 1704 |
| 688 | 8 | A7 | 0.246 | 0.674 | 0.356 | 1120 | 4546 | 1777 | 2636 | 318 | 896 |
| 689 | 8 | B7 | 0.236 | 0.665 | 0.223 | 1176 | 4978 | 2094 | 3147 | 366 | 1642 |
| 690 | 8 | C7 | 0.291 | 0.706 | 0.256 | 1457 | 5006 | 2234 | 3163 | 483 | 1889 |
| 691 | 8 | D7 | 0.289 | 0.796 | 0.244 | 1343 | 4651 | 2259 | 2837 | 411 | 1685 |
| 692 | 8 | E7 | 0.304 | 0.801 | 0.222 | 1347 | 4431 | 2712 | 3386 | 250 | 1129 |
| 693 | 8 | F7 | 0.314 | 0.594 | 0.251 | 1474 | 4698 | 1862 | 3135 | 430 | 1714 |
| 694 | 8 | G7 | 0.305 | 0.882 | 0.243 | 1466 | 4810 | 2200 | 2493 | 513 | 2114 |
| 695 | 8 | H7 | 0.273 | 0.730 | −0.067 | 1164 | 4272 | 1511 | 2070 | −71 | 1063 |
| 696 | 8 | A8 | 0.234 | 0.627 | 0.168 | 1238 | 5282 | 1842 | 2935 | 76 | 456 |
| 697 | 8 | B8 | 0.239 | 0.622 | 0.255 | 1297 | 5419 | 2210 | 3553 | 357 | 1398 |
| 698 | 8 | C8 | 0.324 | 0.768 | 0.228 | 1365 | 4219 | 1832 | 2386 | 342 | 1496 |
| 699 | 8 | D8 | 0.274 | 0.689 | 0.276 | 1325 | 4828 | 2004 | 2907 | 392 | 1418 |
| 700 | 8 | E8 | 0.518 | 1.904 | 1.780 | 2420 | 4668 | 8694 | 4566 | 3410 | 1916 |
| 701 | 8 | F8 | 0.266 | 0.699 | 0.221 | 1362 | 5112 | 2331 | 3336 | 378 | 1707 |
| 702 | 8 | G8 | 0.944 | 1.837 | 1.026 | 4229 | 4479 | 5257 | 2861 | 1717 | 1674 |
| 703 | 8 | H8 | 0.345 | 0.400 | 0.295 | 732 | 2123 | 2606 | 6511 | 466 | 1577 |
| 704 | 8 | A9 | 0.263 | 0.820 | 0.260 | 1218 | 4632 | 1771 | 2161 | 361 | 1387 |
| 705 | 8 | B9 | 0.285 | 0.604 | 0.257 | 1583 | 5551 | 1821 | 3014 | 372 | 1446 |
| 706 | 8 | C9 | 0.286 | 0.663 | 0.299 | 1527 | 5344 | 2004 | 3024 | 410 | 1369 |
| 707 | 8 | D9 | 0.260 | 0.658 | 0.291 | 1128 | 4340 | 1787 | 2717 | 272 | 935 |
| 708 | 8 | E9 | 0.257 | 0.592 | 0.270 | 1125 | 4381 | 1712 | 2894 | 207 | 767 |
| 709 | 8 | F9 | 0.749 | 1.219 | 1.009 | 4019 | 5366 | 5302 | 4350 | 1332 | 1320 |
| 710 | 8 | G9 | 0.313 | 0.954 | 0.716 | 1956 | 6240 | 3903 | 4091 | 1092 | 1525 |
| 711 | 8 | H9 | 0.288 | 0.765 | 0.277 | 1412 | 4899 | 2450 | 3204 | 462 | 1669 |

| | | | | | | | | | |
|---|---|---|---|---|---|---|---|---|---|
| Averages of all wells | 0.323 | 0.756 | 0.351 | 2734 | 7112 | 3496 | 5514 | 867 | 2472 |
| Standard deviations (SD) | 0.139 | 0.285 | 0.282 | 7433 | 11172 | 6043 | 10319 | 1882 | 4684 |
| Averages of non-fluorescent wells | 0.293 | 0.737 | 0.296 | 1442 | 4944 | 2185 | 2989 | 409 | 1369 |
| SD of non-fluorescent wells | 0.062 | 0.113 | 0.132 | 333 | 597 | 438 | 549 | 220 | 410 |

What is claimed is:

1. A method for discovering compounds that modulate metabolite transport across cell membranes comprising the steps of
(a) trapping a probe in cells;
(b) contacting the cells in (a) with a test compound or a mixture of test compounds;
(c) measuring the fluorescence response of the trapped probe in the absence of added unbound metabolite;
(d) adding a defined level of unbound metabolite to the cells plus test compound or mixtures of test compounds of step (b);
(e) measuring the fluorescence response of the trapped probe in the presence of the test compounds and unbound metabolite;
(f) adding a macromolecule that provides a sink for metabolite and thereby reduces unbound metabolite to levels indistinguishable from those in (c);
(g) measuring the fluorescence response of the trapped probe in the presence of the test compounds and absence of unbound metabolite; and
(h) determining a change in influx and/or efflux and/or intracellular level or magnitude of the transported metabolite by comparing the fluorescence response measured in steps (c), (e), and (g), thereby identifying a potential modulator from the test compounds or mixture of test compounds.

2. The method of claim 1, wherein the probe is trapped in the cells by a method selected from the group consisting of electroporation, use of lipid or peptide transfection reagents, and mechanical membrane disruption as in scrape, scratch, bead, or syringe loading.

3. The method of claim 1, wherein the cells are dispersed in multi-well plates and the fluorescence measurements are performed using a high throughput plate scanning fluorometer or microscope.

4. The method of claim 1, wherein the potential modulator discovered using a mixture of test compounds is then re-measured using the individual test compounds in the mixture.

5. The method of claim 1, wherein the potential modulator is tested to determine if it affects the metabolite influx rate constants by measuring an influx time course of the metabolite.

6. The method of claim 1, wherein the potential modulator is tested to determine if it affects the metabolite efflux rate constants by measuring an efflux time course of the metabolite.

7. The method of claim 1, wherein the potential modulator is tested to determine if it affects the intracellular level or magnitude of metabolite transport by measuring the ratio of the intracellular to extracellular unbound metabolite concentrations.

8. The method of claim 1, wherein the potential modulator is tested to determine if it is fluorescent and whether its fluorescence interferes with the probe fluorescence.

9. A method for discovering compounds that inhibit metabolite transport across cell membranes comprising the steps of:
(a) adding cells, test compounds, mixtures of test compounds and combinations thereof to the wells of a multi-well plate;
(b) adding probe to the wells of (a);
(c) measuring the fluorescence response of each probe in each well in the absence of added unbound metabolite, wherein the fluorescence responses are measured under one or more of the following conditions:
(i) the fluorescence response of the probe;
(ii) the fluorescence response of the probe+cell,
(iii) the fluorescence response of the probe+test compound or mixture of test compounds,
(iv) the fluorescence response of the probe+cell+test compound or mixture of test compounds;
(d) adding a composition comprising a weakly buffered complex of the unbound metabolite and a carrier macromolecule to each well so that the level of unbound metabolite decreases in wells with cells that do not contain test compounds, (c)(ii), as compared to wells without cells, (c)(i), (c)(iii);
(e) measuring the fluorescence response of each probe in each well in the presence of added unbound metabolite from step (d), wherein the fluorescence responses are measured under one or more of the following conditions:
(i) the fluorescence response of the probe+unbound metabolite;
(ii) the fluorescence response of the probe+cell+unbound metabolite,
(iii) the fluorescence response of the probe+test compound or mixture of test compounds+unbound metabolite,
(iv) the fluorescence response of the probe+cell+test compound or mixture of test compounds+unbound metabolite;
(f) comparing the fluorescence responses of step (c) to the fluorescence response of step (e), thereby identifying potential modulators.

10. The method of claim 1 or 9 wherein, the potential modulator is tested to determine if the test compound interacts with the probe and either blocks the unbound metabolite's interaction with the probe or induces a probe response or changes the probe response to the unbound metabolite.

11. The method of claim 1 or 9, wherein the potential modulator is tested to determine whether the test compound interacts with the carrier macromolecule so as to alter the unbound metabolite-carrier interaction and/or alter the interaction of the test compound with the unbound metabolite transporter

12. The method of claim 1 or 9, wherein the potential modulator is tested to determine whether the test compound affects cellular lipolysis and/or unbound metabolite metabolism in such a way as to have the appearance of affecting unbound metabolite transport.

13. The method of claim 1 or 9, wherein the probe is ADIFAB and the unbound metabolite is FFAu.
